(19) United States
(12) Patent Application Publication
(10) Pub. No.: US 2026/0069861 A1
(43) Pub. Date: Mar. 12, 2026

(54) A Physiotherapy Device Having Dual-Function Floating Electrodes

(71) Applicant: SHENZHEN NUON MEDICAL EQUIPMENT CO., LTD., Shenzhen (CN)

(72) Inventors: Alain Dijkstra, Amstelveen (NL); LI XIANG, Shenzhen (CN); Tang Yuanzhou, Shenzhen (CN); Chen Kunjie, Shenzhen (CN); Lin Jiaming, Shenzhen (CN)

(73) Assignee: SHENZHEN NUON MEDICAL EQUIPMENT CO., LTD., Shenzhen (CN)

(21) Appl. No.: 19/319,802

(22) Filed: Sep. 5, 2025

(30) Foreign Application Priority Data
Sep. 6, 2024 (CN) 2024221988621
Oct. 21, 2024 (CN) 2024114705957
Oct. 21, 2024 (CN) 2024225417959
Oct. 21, 2024 (CN) 2024225477146

(51) Int. Cl. A61N 1/32; A61N 1/04

(52) U.S. Cl. CPC A61N 1/328; A61N 1/0476

(57) ABSTRACT

The present invention relates to a multifunctional physiotherapy device configured to deliver therapeutic treatments to facial and body skin surfaces. The device comprises a housing defining an application surface, a pair of floating electrodes mounted on elastic members, and a circuit operable to provide microcurrent stimulation. The floating structure enables the electrodes to conform to varying skin contours while simultaneously functioning as charging contacts when engaged with a corresponding interface, thereby combining therapy and charging in a shared element. In certain embodiments, the device incorporates a removable cap containing a power storage unit and output electrodes, enabling portable charging and controlled alignment with the input electrodes. Additional therapeutic features such as temperature regulation, phototherapy, or liquid dispensing may be integrated, thereby providing a compact, ergonomic, and versatile physiotherapy system suitable for skincare and wellness applications across different body areas.

### TECHNICAL FIELD

The present invention relates to the field of physiotherapy and cosmetic treatment devices, and more particularly, to a multi-functional device comprising floating electrodes that perform both microcurrent therapy and charging functions. The invention further relates to devices capable of improving electrode-skin contact, enhancing therapeutic efficiency, and providing an integrated charging mechanism suitable for use on the face and other parts of the human body.

### BACKGROUND

Various types of physiotherapy and cosmetic devices are currently available in the art. Some devices provide microcurrent stimulation, wherein two electrodes, positive and negative, are electrically connected to an internal circuit board. When these electrodes contact the skin, the circuit loop is completed through the skin, enabling therapeutic microcurrent delivery. However, due to manufacturing and assembly tolerances, the precise distance between the electrodes and the circuit board is difficult to control. As a result, reliable contact and electrical connection are not always achieved, which can cause device malfunction and increase the product rejection rate.

Other physiotherapy devices employ therapeutic mechanisms such as LED phototherapy or microcurrent therapy arranged on an application surface intended to contact the skin. Although such devices may provide therapeutic benefit, the electrodes or skin-contact interfaces are typically fixed and rigid. This rigid configuration prevents the electrodes from adapting to the natural curvature and irregularities of the human skin. As a result, electrode-skin adhesion is often inadequate, leading to uneven contact, reduced current conduction or light exposure, and diminished overall therapeutic effectiveness.

In addition, some cosmetic devices integrate therapeutic modules, such as massage units or light therapy elements, into cosmetic containers. These devices allow users to perform therapy while applying cosmetic products. However, such therapeutic modules typically rely on an internal battery that requires charging through a separate dedicated charging device. Since the charging device and cosmetic container are independent components, the charger may be misplaced or difficult to locate when needed, resulting in user inconvenience and interrupted therapy sessions.

Accordingly, the above existing solutions suffer from at least three limitations: (i) difficulty ensuring consistent electrode-circuit connection in microcurrent devices, (ii) poor adhesion between fixed applicator surfaces and irregular skin, and (iii) inconvenience caused by the need for separate charging devices in cosmetic-therapy combinations.

The present invention addresses these deficiencies by providing a multi-functional physiotherapy device with an applicator head having a pair of floating electrodes disposed on its application surface. These electrodes are designed to perform a dual role: acting as charging electrodes when aligned with corresponding output electrodes on a detachable cap, and serving as microcurrent electrodes when in contact with the skin. Each floating electrode is mounted within a mounting hole and supported by an elastic member such as a spring, enabling the electrode to move inward and outward relative to the applicator head. This floating structure allows the electrodes to conform to skin contours under applied pressure, thereby improving adhesion and enhancing therapeutic efficiency. Furthermore, the integration of charging through the cap eliminates the reliance on separate charging accessories, improving convenience and usability.

### OBJECTS OF THE INVENTION

Some of the objects of the invention are as follows:

An object of the present invention is to provide a multifunctional physiotherapy device with floating electrodes mounted on elastic members, capable of conforming to the natural curvature of the skin for improved adhesion and therapeutic efficiency.

Another object of the present invention is to provide a physiotherapy device wherein the same electrodes perform a dual role, functioning both as microcurrent therapy electrodes during treatment and as charging electrodes when aligned with corresponding output electrodes on a detachable cap.

Another object of the present invention is to provide a physiotherapy device with electrodes supported by springs, flexible arms, or elastic structures, allowing inward and outward displacement relative to the application surface, thereby enhancing comfort, adaptability, and stability during use.

Another object of the present invention is to integrate a detachable cap with the device, wherein the cap contains output electrodes that engage with the floating electrodes for direct charging, eliminating the need for separate charging accessories and improving portability.

Another object of the present invention is to provide a physiotherapy device wherein the detachable cap may further include auxiliary therapy modules such as light therapy sources, heating or cooling elements, or vibration units to deliver multimodal physiotherapy.

Another object of the present invention is to enhance hygiene and safety by enabling disinfection of the electrodes through germicidal light or similar sterilization means when the device is placed in the charging cap or base.

Another object of the present invention is to provide a physiotherapy device with an optional liquid outlet disposed between the electrodes on the application surface, enabling simultaneous dispensing of skincare or therapeutic solutions in coordination with electrical or thermal therapy.

Another object of the present invention is to provide a physiotherapy device that is compact, ergonomic, and suitable for use on any part of the body, including but not limited to the face, scalp, neck, arms, and legs, thereby extending its therapeutic applicability.

Another object of the present invention is to provide a versatile physiotherapy device that may be manufactured in different shapes and sizes, allowing adaptation for handheld personal use, portable travel models, or larger therapeutic instruments for clinical applications.

### SUMMARY OF THE INVENTION

According to a first aspect of the present invention, a physiotherapy device is provided. The physiotherapy device comprising: a housing defining an application surface configured to contact skin; a pair of electrodes mounted on the application surface, each electrode being movably supported by an elastic member to allow axial displacement relative to the housing; a circuit configured to deliver therapeutic current to the electrodes; and a control mechanism operable to selectively enable a microcurrent therapy circuit, wherein the electrodes are configured to conform to the contour of a skin surface during use.

In one embodiment of the invention, each elastic member comprises a spring, a metal strip, or a flexible conductive arm configured to provide resilient displacement of the electrode relative to the housing.

In one embodiment of the invention, the pair of input electrodes is a floating electrode movably supported within a mounting cavity and resiliently biased by the elastic member to maintain continuous contact with the uneven skin surface.

In one embodiment of the invention, the electrodes each comprise a top wall and a side wall extending along a periphery of the top wall, the side wall defining a positional stop for maintaining the electrode in the housing.

In one embodiment of the invention, the top wall of the electrode has a meniscus-shaped or curved surface to enhance skin contact, and the elastic member is positioned adjacent to a corner of the electrode.

In one embodiment of the invention, the physiotherapy device further comprising a coating surface and a liquid outlet formed on the housing, the liquid outlet being positioned between the pair of electrodes such that liquid applied from the outlet is concurrently delivered with microcurrent stimulation.

In one embodiment of the invention, the control mechanism comprises a manual button, touch-sensitive switch, or sensor configured to selectively enable the microcurrent therapy circuit.

In one embodiment of the invention, the pair of input electrodes is configured to function as charging electrodes when engaged with a charging base or a cap.

According to a second aspect of the present invention, a physiotherapy device is provided. The physiotherapy device comprising: a housing; a battery disposed within the housing; a circuit board electrically connected to the battery; a pair of input electrodes exposed outside the housing; wherein the battery, the circuit board, and the pair of input electrodes are electrically connected to form a microcurrent circuit and a charging circuit, the microcurrent circuit and the charging circuit being independent from one another; and wherein the microcurrent circuit is configured to deliver microcurrent stimulation and the charging circuit is configured to charge the battery.

In one embodiment of the invention, the physiotherapy device further comprising a trigger switch connected to the circuit board, the trigger switch is configured to generate a trigger signal to the circuit board to allow the microcurrent circuit to enter in a connected state while simultaneously controlling the charging circuit to enter in a disconnected state.

In one embodiment of the invention, the physiotherapy device further comprising a first switch connected to the microcurrent circuit and a second switch connected to the charging circuit; and wherein the circuit board is configured to control the first switch in a connected state and the second switch in a disconnected state upon receiving a trigger signal.

In one embodiment of the invention, the physiotherapy device further comprising a sensor to detect at least one parameter of a user's skin; and wherein the circuit board is configured to control the microcurrent circuit in a connected state and the charging circuit in a disconnected state when the at least one parameter exceeds above a threshold value

In one embodiment of the invention, the physiotherapy device further comprising one or more stimulation element selected from a group consisting of but is not limited to a phototherapy lamp, a vibration motor, a hot compress module, and a cold compress module.

In one embodiment of the invention, the housing defines an applicator surface and a mounting cavity for accommodating the pair of input electrodes, and the pair of input electrodes is movably supported by an clastic conductor, forming floating electrodes configured to maintain contact with the skin.

In one embodiment of the invention, the physiotherapy device further comprising a detachable cap including a pair of output electrodes, wherein the pair of input electrodes engages with the pair of output electrodes when the cap is mounted to enable charging of the battery.

In one embodiment of the invention, the physiotherapy device further comprising a liquid container defining a storage cavity for a skin care product, wherein the housing is attachable to the liquid container, and the pair of input electrodes is disposed on an applicator surface for simultaneous delivery of the skin care product and microcurrent therapy.

According to a third aspect of the present invention, a method for delivering physiotherapy or skin care treatment to a user is provided. The method comprising: providing a physiotherapy device comprising a housing defining an application surface, a pair of input electrodes mounted on the application surface, and a circuit electrically connected to the pair of input electrodes; placing the application surface in contact with the skin of the user; enabling a microcurrent therapy circuit via a control mechanism; causing the pair of input electrodes to conform to the contour of the user's skin; and delivering therapeutic current through the pair of input electrodes to the skin.

In one embodiment of the invention, the pair of input electrodes is movable, supported by an elastic member comprising a spring, a metal strip, or a flexible conductive arm, such that the electrodes are axially displaceable relative to the housing.

In one embodiment of the invention, the method further comprising activating the control mechanism via a manual button, touch-sensitive switch, or sensor to selectively enable the microcurrent therapy circuit.

In one embodiment of the invention, the method further comprising establishing a charging circuit when the electrode engages with a pair of output electrodes of a detachable cap or a charging base, independently of the microcurrent therapy.

In one embodiment of the invention, the method further comprising moving the electrode axially via an elastic conductor such that the electrode maintains continuous contact with the uneven skin surface during treatment.

In this way, the present invention integrates therapeutic stimulation, liquid dispensing, charging, and auxiliary modalities (light, heat/cold, vibration) into a compact multifunctional device. By employing movably mounted floating electrodes that serve dual purposes as both therapy and charging electrodes, the invention overcomes the limitations of conventional rigid therapy applicators and separate charging systems, thereby improving usability, therapeutic efficiency, and portability.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term “several” means more than one, unless otherwise specified.

In the context of the specification, the term “parallel” encompasses both parallel and substantially parallel (nearly parallel) orientations, unless otherwise specified.

In the context of the specification, the term “LED” refers to one or more light-emitting diode (LED) elements that are electrically connected and configured to emit light of specific wavelengths (including visible to IR wavelengths) suitable for therapeutic purposes. The LED element or module may include drive circuitry, heat dissipation structures, and optical elements such as lenses or diffusers to control light distribution.

In the context of this specification, terms like “light,” “radiation,” “irradiation,” “emission,” and “illumination,” etc., refer to electromagnetic radiation in frequency ranges varying from the visible frequencies to Infrared (IR) frequencies and wavelengths, wherein the range is inclusive of visible light, and IR frequencies and wavelengths. Preferably, it refers to low-level electromagnetic radiation of low-level red and near-infrared (NIR) light.

In the context of the specification, the term “light source” or “phototherapy source,” etc., refers to a source emitting coherent laser light, or light-emitting diodes (“LEDs”). The term “light therapy” refers to light generated from any of the sources, such as lasers, LED sources, or Super luminous diodes (“SLD”), or Organic light-emitting diodes (OLED).

In the context of the specification, the term “lamp board” refers to a printed circuit board or similar substrate on which one or more light sources are mounted and electrically connected to a power source, such as a rechargeable battery.

In the context of the specification, “Light Emitting Diodes (LEDs)” refer to semiconductor diodes capable of emitting electromagnetic radiation when supplied with an electric current. The LEDs may also include UV-emitting diodes specifically selected for antimicrobial action in disinfection modules integrated into the multifunctional physiotherapy comb device.

In the context of the specification, the term “electrode” refers to a conductive element configured to contact skin and deliver or receive electrical signals. Unless otherwise specified, electrodes described herein may perform a dual function: (i) serving as therapy electrodes for delivering microcurrent or other electrical stimulation to a skin surface; and (ii) serving as charging electrodes when engaged with a corresponding charging interface or cover body. Electrodes may be rigid, flexible, or supported in a floating configuration by an elastic or resilient member that permits axial movement relative to the device housing, thereby enhancing skin conformity and maintaining reliable charging engagement.

In the context of the specification, the term “control mechanism” refers to any manual, electrical, or sensor-based input that allows the user or device to selectively activate or deactivate therapy functions, charging functions, or both. This may include physical switches, capacitive touch buttons, or automatic detection circuits.

In the context of the specification, the term “power storage unit” refers to a rechargeable battery, capacitor, or equivalent device configured to supply electrical energy to the physiotherapy device. The power storage unit may be housed within a removable cap or cover body, enabling the cap to operate as a portable power bank.

In the context of the specification, the term “vibration module” refers to any electromechanical arrangement configured to impart oscillatory motion to a portion of the device. This may include eccentric rotating mass motors, linear resonant actuators, piezoelectric actuators, or other vibration-generating mechanisms. Vibration may be employed to enhance massage, improve liquid penetration, or stimulate circulation.

In the context of the specification, the term “thermal module” or “heating/cooling module” refers to a temperature control system integrated into the device. Such a module may be configured to provide controlled heating, cooling, or alternating thermal cycles to the skin surface. Heating elements may include resistive heaters, infrared emitters, or thermoelectric (Peltier) devices, while cooling may be provided by Peltier elements, heat sinks, or fluid-based cooling systems.

In the context of the specification, the term “liquid dispensing module” refers to a structure enabling controlled delivery of a liquid, gel, or semi-liquid substance from a reservoir to the user's skin. The dispensing module may include valves, rollers, hollow comb teeth, porous applicators, or micro-orifices. In some embodiments, dispensing may be pressure-sensitive, roller-actuated, or electronically controlled, and may operate simultaneously with microcurrent therapy or phototherapy.

In the context of the specification, the term “therapy module” refers broadly to any combination of the above-described components, including electrodes, light sources, vibration modules, thermal modules, or liquid dispensing elements, integrated into a single device or into interchangeable attachments.

In the context of this specification, terms like “light,” “radiation,” “irradiation,” “emission,” and “illumination,” etc., refer to electromagnetic radiation in frequency ranges varying from the visible frequencies to Infrared (IR) frequencies and wavelengths, wherein the range is inclusive of visible light, and IR frequencies and wavelengths. Preferably, it refers to low-level electromagnetic radiation of low-level red and near-infrared (NIR) light. It is to be noted here that IR radiation can be categorised into several categories according to respective wavelength ranges, which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm), and Far IR (15-1000 μm). In this regard, light application is at relatively low energy densities, typically below about 500 mW, as compared to other forms of laser therapy that are used for ablation, cutting, and thermally coagulating tissue. In some instances, electromagnetic radiation can also be in wavelengths in the blue or ultraviolet regions, especially for the treatment of conditions that occur at the skin surface, such as psoriasis or infection.

In the context of the specification, the term “light source” or “phototherapy source,” etc., refers to a source emitting coherent laser light, or light-emitting diodes (“LEDs”). The term “light therapy” refers to light generated from any of the sources, such as lasers, LED sources, or Super luminous diodes (“SLD”), or Organic light-emitting diodes (OLED).

In the context of the specification, the term “lamp board” refers to a printed circuit board or similar substrate on which one or more light sources are mounted and electrically connected to a power source, such as a rechargeable battery.

In the context of the specification, the term “light source” refers to any active component, such as a light-emitting diode (LED), capable of emitting phototherapy light (e.g., red, infrared, or blue) toward the scalp.

In the context of the specification, the term “light transmission hole” refers to an aperture formed in the housing of the second rotating arm, aligned with the light source to allow therapeutic light to exit the comb and reach the scalp.

In the context of the specification, the term “light transmission plate” refers to a transparent or translucent cover positioned over the light transmission hole to protect internal components while allowing light to pass through.

In the context of the specification, the term “pressing switch” refers to a mechanical actuator disposed on the surface of the comb and configured to trigger an internal switching device that activates the lamp board or associated electronics.

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colours), Surface Mount Technology (SMT) LEDs, Bi-colour LEDs, Pulse Width Modulated RGB (Red-Green-Blue) LEDs, and high-power LEDs, among others. The LEDs may also include UV-emitting diodes specifically selected for antimicrobial action in disinfection modules integrated into the multifunctional physiotherapy comb device.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminium, Boron, Zinc Selenide, etc., in pure form or doped with elements such as Aluminium and Indium, etc. For example, red and amber colours are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider colour gamut. White and other colored lightings may also be produced using phosphor coatings such as Yttrium Aluminium Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety by reference.

In the case of a stimulation element being an electrode, the stimulation element may be embodied as an open-ended conductor. The electrode may then be able to provide Transcutaneous Electrical Nerve Stimulation (TENS), Electronic Muscle Stimulation (EMS), and Microcurrent Electrical Therapy (MET) to the target surfaces. TENS therapy uses low-voltage currents to provide pain relief. Electrical impulses are delivered through electrodes placed on the surface of the body of the user.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1 illustrates a perspective view of a physiotherapy device, in accordance with an embodiment of the present invention.

FIG. 2 illustrates a front perspective view of an applicator portion of the physiotherapy device, in accordance with an embodiment of the present invention.

FIG. 3 illustrates an exploded view of the housing of the physiotherapy device, in accordance with an embodiment of the present invention.

FIG. 4 shows a cross-sectional view of the housing of the physiotherapy device, in accordance with an embodiment of the present invention.

FIG. 5 illustrates a cross-sectional view of a cover of the housing, in accordance with an embodiment of the present invention.

FIG. 6 illustrates an internal perspective view of a base of housing, in accordance with an embodiment of the present invention.

FIG. 7 illustrates an exploded view of a cap of the physiotherapy device, in accordance with an embodiment of the present invention

FIG. 8 illustrates a perspective view of another configuration of a physiotherapy device, in accordance with an embodiment of the present invention.

FIG. 9 illustrates a perspective view of the physiotherapy device, in accordance with an embodiment of the present invention.

FIG. 10 illustrates an exploded view of a cap body of the physiotherapy device, in accordance with an embodiment of the present invention.

FIG. 11 illustrates an exploded view of a container body of the physiotherapy device, in accordance with an embodiment of the present invention.

FIG. 12 illustrates an exploded view of a charging device of the physiotherapy device, in accordance with an embodiment of the present invention.

FIG. 13 illustrates a front view of the physiotherapy device, in accordance with an embodiment of the present invention.

FIG. 14A illustrates a block diagram representing the working of the microcurrent circuit using a trigger button in the physiotherapy device, in accordance with an embodiment of the present invention.

FIG. 14B illustrates a block diagram representing the working of the microcurrent circuit using a sensor in the physiotherapy device, in accordance with an embodiment of the present invention.

FIG. 15A illustrates a block diagram representing the working of the charging circuit using a trigger button in the physiotherapy device, in accordance with an embodiment of the present invention.

FIG. 15B illustrates a block diagram representing the working of the charging circuit using a sensor in the physiotherapy device, in accordance with an embodiment of the present invention.

FIG. 16A and FIG. 16B illustrates a perspective view of another configuration of a physiotherapy device, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practised. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

Embodiments of the present invention provide a multi-functional physiotherapy device comprising a housing defining an application surface configured to contact the skin of a user. A pair of electrodes is mounted on the application surface, each disposed within a mounting hole of the housing and movably supported by an elastic member such as a spring, a flexible conductive strip, or an elastomeric element. This floating configuration permits the electrodes to move axially inward or outward relative to the housing under applied pressure, thereby enabling the electrodes to conform to the irregular contours of human skin, increasing contact adhesion, and ensuring a more uniform distribution of current during therapy. The electrodes are electrically connected to a circuit disposed within the housing, which may include a printed circuit board configured to generate therapeutic microcurrent waveforms suitable for skin stimulation. A control mechanism, which may include a push button, capacitive touch switch, or sensor, is operable to selectively activate or deactivate the microcurrent therapy circuit. During operation, when the electrodes contact the skin, they form a closed microcurrent loop through the skin and the circuit board.

In certain embodiments, the electrodes serve a dual purpose as charging contacts. When aligned with a corresponding charging interface, the electrodes receive electrical power to recharge an internal power storage unit disposed within the housing, thereby integrating therapeutic and charging functions into a single electrode pair for simplified device structure and enhanced user convenience. In another embodiment, the physiotherapy device is integrated with a cosmetic or skincare container. The container body defines a storage space for holding a liquid or semi-liquid substance, such as lotion, serum, or gel. A cap is removably attached to the container body, with the cap housing a therapy module including a pair of electrodes operable to deliver microcurrent stimulation. In this configuration, the user may remove, rotate, or directly use the cap to apply the therapy module to the skin while simultaneously dispensing the skincare substance. A liquid outlet may be formed between the electrodes, such that the substance is delivered to the skin concurrently with microcurrent stimulation, thereby improving absorption and therapeutic benefit.

The therapy module housed in the cap may optionally include additional stimulation elements. For example, a temperature element such as a heating resistor, cooling element, or Peltier device may be thermally coupled to the electrodes to provide hot or cold compress therapy. In another example, a light-emitting module, such as an LED array or laser diode, may be provided to emit therapeutic light through a light-transmitting portion of the cap, allowing phototherapy in the red, blue, or infrared spectrum. A vibration module may also be incorporated to provide massage stimulation. These features may be activated individually or in combination under the control of an electronic circuit.

In further embodiments, the device comprises a housing with an application surface bearing the input electrodes electrically connected to a circuit board, and a removable cap body configured to cover the housing. The cap may contain a power storage unit, such as a rechargeable lithium-ion battery, and output electrodes electrically coupled to the power storage unit. When the cap is attached, the output electrodes align and electrically connect with the input electrodes on the housing to transfer electrical energy for charging. When removed, the input electrodes are exposed for direct application to the skin. The cap thus functions as a portable power bank and may include a USB charging port, wireless charging coil, or magnetic charging interface to recharge its internal power storage unit, allowing convenient portable charging.

The device may further include automatic switching logic to distinguish between therapy and charging modes. For example, the control circuit may measure resistance values to differentiate between skin contact and metallic contact with the charging electrodes, or a position sensor may detect whether the cap is engaged with the housing, thereby selecting the appropriate mode. The electrodes may also function as thermal conduction elements, with heat or cold transferred directly from a heating resistor or thermoelectric module to the user's skin. The electrodes may be formed of or coated with thermally conductive and biocompatible materials. To maintain hygiene, the charging device or container body may incorporate an ultraviolet light source, such as a UV-C LED, positioned to irradiate the electrodes when the cap is docked, optionally with a transparent window to allow radiation while shielding sensitive components.

The cap may be ergonomically designed with a first portion coaxially aligned with the bottle opening and a second portion disposed eccentrically relative to the first portion. The second portion accommodates the therapy module, output electrodes, and power storage unit, increasing the effective rotation arm length to reduce torque during attachment or removal. Complementary stepped surfaces and limiting ribs on the cap and container body ensure proper electrode alignment, restrict over-rotation, and improve scaling integrity.

The physiotherapy device provides versatile functionality, including microcurrent stimulation with improved skin adhesion, integration of charging functionality into therapeutic electrodes, combined application of skincare products and therapy, and additional treatment modalities such as phototherapy, massage, and thermal therapy. The cap functions as a portable power bank, enhancing user convenience by eliminating the reliance on separate charging accessories. The housing may include a pair of elastic conductors extending between input electrodes and conductive connectors fixed to a circuit board, enabling resilient electrical pathways. The input electrodes may be configured as floating electrodes supported by the elastic conductor, maintaining consistent skin contact under pressure while ensuring reliable conduction during therapy and charging operations.

The physiotherapy device may also comprise a liquid container and a housing that together provide storage, dispensing, and integrated therapeutic functions. The housing may include a first portion rotatably connected to the bottle opening and a second eccentric portion accommodating electronic components, forming stepped surfaces with corresponding portions of the liquid container. A control mechanism allows manual or automatic switching between microcurrent and charging modes. Input electrodes may function as heat transfer elements coupled to a temperature element, providing hot or cold compress therapy. A stimulation surface on the housing and a corresponding charging surface on a charging device enable simultaneous electrical conduction and rotational limiting when mated.

This design minimizes the number of electrodes required, simplifies the overall structure, reduces production complexity and material costs, enhances sealing and alignment, and ensures reliable microcurrent stimulation, thermal therapy, phototherapy, and vibration therapy. The integrated charging design enables timely and consistent recharging of the housing's power storage unit without requiring separate charging accessories, while UV sterilization ensures hygienic maintenance of electrodes. The device is thus capable of providing a comprehensive, multi-functional physiotherapy experience in a compact and convenient form factor.

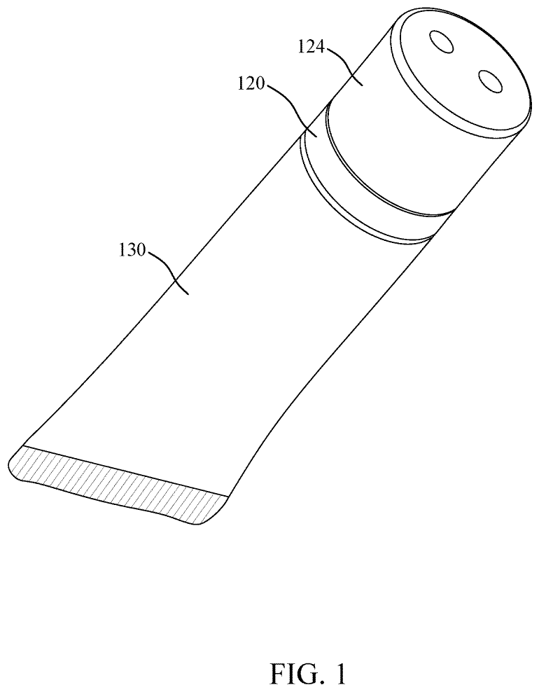

Referring to FIGS. 1 to 7 illustrates a configuration of a physiotherapy device. The physiotherapy device 200 comprises a cap 124, a housing 100, a base 120, and a liquid container 130. The housing 100 comprises a pair of electrodes, a pair of elastic conductors 134, a pair of conductive connectors 140, a first battery 246a, and a circuit board 146. Each input electrode 122 is operatively associated with a corresponding elastic conductor 134 and conductive connector 140 in a one-to-one manner.

The housing 100 is provided with two mounting holes 112, into which the input electrodes 122 are mounted such that they are exposed for direct contact with the user's skin. The elastic conductors 134, conductive connectors 140, and the circuit board 146 are accommodated within the housing 100. The conductive connectors 140 are securely fixed to, and electrically coupled with, the circuit board 146.

Each elastic conductor 134 extends between an input electrode 122 and its corresponding conductive connector 140, maintaining elastic abutment at both ends. This configuration enables the elastic conductor 134 to serve as a resilient conductive pathway between the input electrode 122 and the conductive connector 140. When both input electrodes 122 are simultaneously in contact with the user's skin, the two input electrodes 122, elastic conductors 134, conductive connectors 140, and the circuit board 146 cooperate to form a closed electrical circuit. In this state, controlled microcurrent stimulation is delivered to the skin, thereby achieving the desired therapeutic effect.

A key advantage of employing the elastic conductor 134 lies in its inherent elasticity, which allows it to automatically compensate for assembly tolerances, dimensional variations, or positional wobble between the input electrode 122 and the conductive connector 140. As a result, the clastic conductor 134 consistently maintains reliable electrical contact with both components. In contrast, the conductive connectors 140, being rigidly secured to the circuit board 146, retain a fixed relative position and provide a stable, low-resistance interface to the circuit board 146.

Through this arrangement, a robust and stable electrical connection is ensured between the input electrode 122 and the circuit board 146 with the help of a first battery 246a positioned right beside the circuit board 146, irrespective of variations in the length or shape of the elastic conductors 134. This structural configuration enhances reliability, ensures consistent microcurrent delivery, and thereby improves the overall user experience of the physiotherapy device 200.

In certain embodiments, the elastic abutment between the elastic conductor 134 and the input electrode 122 eliminates the need for welding or permanent fixation. This facilitates both assembly and disassembly of the device, thereby simplifying manufacturing and enabling convenient repair or replacement of individual components as needed.

In some embodiments, the input electrode 122 comprises a top wall 128 and a side wall 152 extending along the periphery of the top wall 128. The top wall 128 is configured to cover the mounting hole 112, while the side wall 152 extends at least partially into the mounting hole 112. The end of the elastic conductor 134 abuts against the underside of the top wall 128 while simultaneously contacting the side wall 152. In this arrangement, the clastic conductor 134 is disposed near the edge of the input electrode 122. The contact between the clastic conductor 134 and the side wall 152 constrains the clastic conductor 134, preventing it from slipping radially beyond the side wall 152 and thereby maintaining reliable abutment with the top wall 128. Structurally, the top wall 128 and side wall 152 together define a lid-like configuration, a cap 124, sealing the mounting hole 112. The seal may be reinforced by a sealing ring or sealant. Furthermore, the side wall 152 extends into the mounting hole 112, serving as a positional stop that prevents both the input electrode 122 and the elastic conductor 134 from radial displacement within the mounting hole 112.

In other embodiments, the input electrode 122 is formed with a groove at its bottom surface, into which the end of the elastic conductor 134 is inserted. This groove provides positional control and ensures reliable placement of the elastic conductor 134 relative to the input electrode 122.

In yet other embodiments, the top wall 128 may be meniscus-shaped, defining two corners 110 at opposite ends. The elastic conductor 134 may be positioned adjacent to one of the corners 110. Owing to the crescent-shaped geometry of the top wall 128, the ends are narrower while the central portion is wider. As a result, the corners 110 provide relatively confined positioning spaces for the elastic conductor 134, allowing the side wall 152 to clamp the conductor securely in place and reduce the risk of undesired movement. Meanwhile, the wider central region of the top wall 128 provides a larger contact surface with the user's skin, enhancing the delivery of electrical stimulation therapy. In alternative embodiments, the top wall 128 may assume a circular, square, or irregular geometry depending on design requirements.

Additionally, a retaining flange 104 may be formed at the distal end of the side wall 152, opposite to the top wall 128. The retaining flange 104 engages with the inner wall of the housing 100, thereby preventing the input electrode 122 from moving upward and disengaging from the mounting hole 112. In some variants, the retaining flange 104 is annular and surrounds the circumference of the mounting hole 112, providing uniform retention.

In further embodiments, the outer surface of the housing 100 defines an application surface 136 and a liquid channel 142. The liquid channel 142 has an inlet connected to a liquid container 130 and an outlet that opens through the application surface 136. The application surface 136 may be concave and arcuate, with the liquid outlet located at its lowest point, thereby promoting controlled liquid flow and retention. Mounting holes 112 for the input electrodes 122 are positioned on opposite sides of the liquid outlet. The concave configuration of the application surface 136 conforms more closely to the natural curvature and irregularities of human skin, making it particularly suitable for application to facial areas such as the chin.

During operation, liquid from the liquid container 130, which may include skincare products such as sunscreen, eye cream, face cream, or essence, flows through the liquid channel 142 and exits via the liquid outlet onto the application surface 136 for application to the skin. When the user's skin contacts the input electrodes 122 during this process, microcurrents are simultaneously delivered. The combination of liquid application and electrical stimulation promotes enhanced absorption of the applied skincare product, thereby improving treatment efficacy.

In some embodiments, the input electrodes 122 are arranged flush with the application surface 136, thereby ensuring complete contact between the liquid outlet and the user's skin. This configuration minimizes residual liquid at the outlet and promotes more efficient transfer of liquid from the device to the skin surface. Alternatively, the input electrodes 122 may protrude slightly from the application surface 136, thereby increasing the likelihood of direct skin contact.

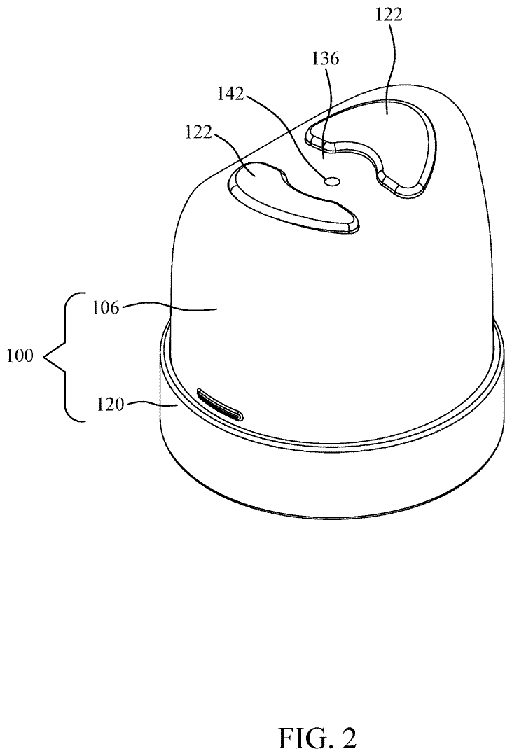

Referring to FIGS. 2, 5, and 6, in an embodiment, the housing 100 comprises an upper portion 106 and a base 120. The upper portion 106 includes the mounting hole 112, while the base 120 covers the end of the upper portion 106 opposite the mounting hole 112. The base 120 is provided with a support member 138, positioned to support the side of the conductive connector 140 facing away from the mounting hole 112. Configuring the housing 100 as two separate parts, the upper portion 106 and the base 120, facilitates independent processing and reduces manufacturing difficulty. Furthermore, the support member 138 prevents the conductive connector 140 from being displaced by the elastic conductor 134, ensuring stable retention by simultaneously clamping the conductive connector 140 between the support member 138 and the elastic conductor 134.

The upper portion 106 generally defines a hollow downwardly-opening structure, with the mounting hole 112 formed on its top surface. The base 120 closes the lower opening of the upper portion 106, thereby sealing the interior cavity to prevent ingress of water, dust, or other contaminants.

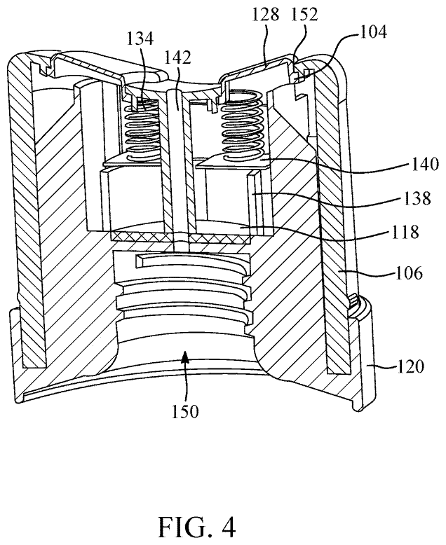

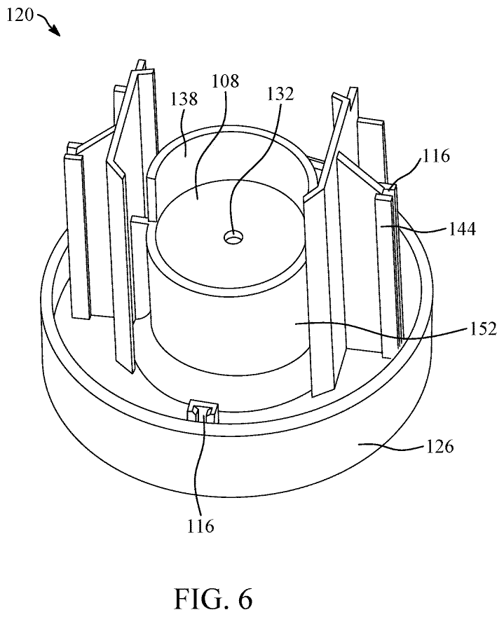

Referring to FIGS. 4 and 6, in an embodiment, the base 120 includes a covering portion 126 and a fixing protrusion 150. The covering portion 126 overlies the lower end of the upper portion 106 and may be disc-shaped or circular. The fixing protrusion 150 extends from the covering portion 126 into the interior space of the upper portion 106. The fixing protrusion 150 is hollow and defines an opening 102, located on the side of the covering portion 126 opposite the upper portion 106, for insertion of the end of the liquid container 130. A communication hole 132 is further defined in the fixing protrusion 150, providing fluid communication between its interior and the liquid channel 142 formed in the upper portion 106.

In certain variants, the inner wall of the fixing protrusion 150 is threaded to mate with a correspondingly threaded end of the liquid container 130. Alternatively, the liquid container 130 may be directly press-fitted into the opening 102 for a secure connection. Once installed, liquid stored in the liquid container 130, such as sunscreen, eye cream, face cream, essence, or similar skincare formulations, flows from the liquid container 130 through the communication hole 132 into the liquid channel 142, and is dispensed through the outlet onto the application surface 136 for direct skin application.

In some embodiments, the support member 138 is integrally formed with the fixing protrusion 150, disposed near its end adjacent to the top wall 128. The surface of the support member 138 opposite the fixing protrusion 150 supports the conductive connector 140, thereby preventing displacement. Alternatively, the support member 138 may be formed on the inner sidewalls 152 of the base 120 or on the inner surface of the upper portion 106.

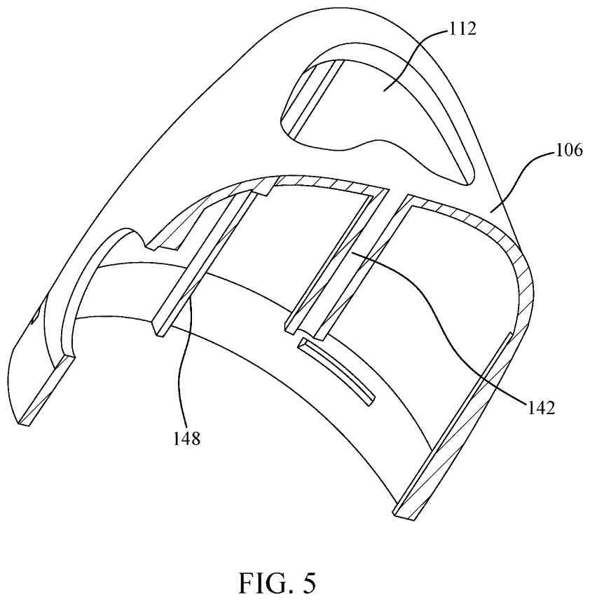

Referring to FIGS. 5 and 6, in an embodiment, the base 120 is concave, facing upward toward the upper portion 106. Specifically, the covering portion 126 assumes a recessed profile that closely accommodates the end of the upper portion 106. The base 120 further includes a raised portion 144 forming a retaining groove 116. The inner wall of the upper portion 106 is provided with a corresponding retaining rib 148. The retaining rib 148 and retaining groove 116 extend along the insertion direction, for example, vertically, such that during assembly, the upper portion 106 is inserted downward into the recessed cavity of the base 120. As the components are engaged, the retaining rib 148 slides along the retaining groove 116, guiding the insertion and preventing rotation of the upper portion 106 relative to the base 120. Once fully assembled, the lower end of the upper portion 106 is housed within the base 120, with the outer circumferential wall of the upper portion 106 in contact with the inner circumferential wall of the base 120. Simultaneously, the retaining rib 148 engages with the retaining groove 116, thereby securing the upper portion 106 in both radial and axial directions. The raised portion 144 additionally reinforces the structural strength of the base 120.

Furthermore, in an embodiment, the fixing protrusion 150 defines a receiving groove 108 on its side facing the mounting hole 112, allowing improved accommodation and positioning of adjacent components.

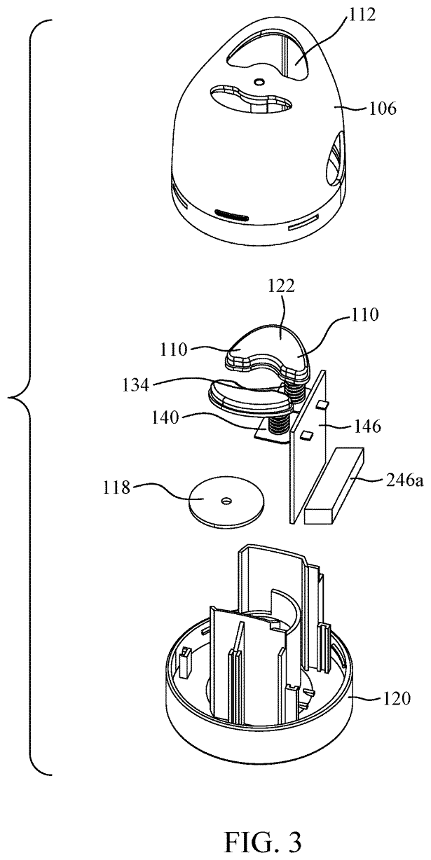

In an embodiment, a communicating hole 132 extends through the bottom of the receiving groove 108. A sealing gasket 118 (see FIG. 3) is disposed within the receiving groove 108. The sealing gasket 118 defines a through hole that aligns with the liquid passage. The upper portion 106 further includes a conduit, within which the liquid channel 142 is formed. The lower end of the conduit abuts against the sealing gasket 118, such that the through hole of the sealing gasket 118 fluidly connects the liquid channel 142 with the communicating hole 132. This configuration enhances sealing reliability and prevents leakage at the junction between the liquid container 130 and the liquid channel 142.

Referring to FIG. 5, in an embodiment, the conductive connector 140 is plate-shaped and arranged facing the mounting hole 112. The plate-shaped configuration increases the contact area with the elastic conductor 134, thereby improving positional stability and ensuring a more reliable conductive connection. In this embodiment, the conductive connector 140 is arranged substantially perpendicular to the circuit board 146, which further stabilizes the structural and electrical connection.

In certain embodiments, the elastic conductor 134 may take the form of a spring, providing elastic resilience in both axial and radial directions. Alternatively, the elastic conductor 134 may be a metal strip, with its two ends bent to form resilient elastic arms capable of abutting against the input electrode 122 and the conductive connector 140, respectively. Both variations achieve the desired function of maintaining elastic abutment and consistent electrical conduction.

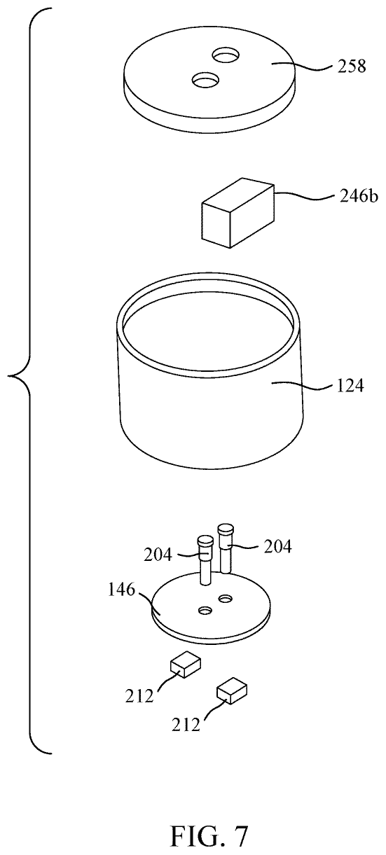

Referring to FIGS. 1 and 7, in some embodiments, the physiotherapy device 200 further comprises a cap 124, which encloses the upper end of the housing 100, thereby covering both the input electrode 122 and the liquid outlet of the liquid channel 142. FIG. 7 illustrates an exploded view of a removable cap 124. The removable cap 124 includes a power receiving terminal or output electrodes 204 configured to receive electrical power from an external electrical power source, either through a direct conducting connection or via electromagnetic induction. The removable cap 124 further comprises a cap body configured to enclose therein a cap circuit board 146. A first charging module 258 (at least two power receiving terminals) is electrically coupled to the circuit board 146 and is configured to receive electrical power from the external power source. The power terminals or output electrodes 204 protrude downward from respective apertures formed in the circuit board 146. Additionally, one or more Ultraviolet (UV) light sources 212 are electrically coupled to the circuit board 146 and are configured to emit UV light upon activation with electrical current.

In an embodiment, after the use of the physiotherapy device 200, the removable cap 124 may be fastened onto the liquid container 130. As a result, the power terminals or output electrodes 204 enclosed within the removable cap 124 make electrical contact with the corresponding charging terminals or input electrodes 122 provided on the upper portion 106 of the housing 100, thereby supplying electrical power from an external power source to the charging terminals or input electrodes 122. Furthermore, when the cap 124 is mounted, the upper portion 106 and one or more stimulation elements may be sterilized by the UV light sources integrated within the removable cap 124, thereby maintaining hygiene and ensuring safe reuse of the device.

The input electrodes 122 serve a dual function of both charging and providing therapeutic stimulation. In an embodiment, the input electrodes 122 also act as therapeutic electrodes for delivering microcurrent stimulation. Specifically, the input electrodes 122, the first battery 246a disposed within the physiotherapy device, and a circuit board 146 disposed within the housing 100 are electrically connected to form a microcurrent circuit. When the input electrodes 122 come into contact with the skin, an electrical path is formed through the skin, thereby closing the microcurrent circuit among the input electrodes 122, the circuit board 146, the first battery 246a, and the skin. This allows microcurrent stimulation to be applied directly to the skin.

In an embodiment, the system further incorporates a detachable cap 124, which includes a second battery 246b configured to operate as an auxiliary battery or power bank. The physiotherapy device and the cap 124 thus each contain separate power storage units: the cap's power storage unit 246b provides auxiliary charging capability, while the device's internal power storage unit 246a powers therapeutic operations.

The input electrodes 122 are configured to participate in a dual charging arrangement. When the input electrodes 122 engage with the output electrodes 204 disposed on the cap 124, a charging circuit is established between the first battery 246a and the second battery 246b. This enables bidirectional charging: the cap can recharge the physiotherapy device, or conversely, the physiotherapy device can recharge the cap, depending on relative charge states. Importantly, this charging mode is active only when the electrodes are not functioning as microcurrent electrodes.

Accordingly, the system employs a shared set of input electrodes 122 that alternately function as charging electrodes or as microcurrent electrodes. The microcurrent circuit and charging circuit are designed as independent, mutually exclusive circuits, such that only one is active at a given time. When the microcurrent circuit is active, the charging circuit is disabled, and vice versa. This architecture ensures both safe operation and efficient use of the shared electrode pair.

When the input electrodes 122 are then placed in contact with the skin, the microcurrent loop is completed, thereby delivering therapeutic microcurrent stimulation. Conversely, when the circuit board 146 does not receive a trigger signal, it defaults to disabling the microcurrent circuit and enabling the charging circuit. In this state, when the input electrodes 122 engage with the output electrodes 204 of the cap 124, a charging loop is formed between the first battery 246a and the second battery 246b, allowing one to replenish the other.

This shared-electrode and dual-battery design provides several advantages: (i) charging and therapy are both achieved using the same pair of electrodes, thereby reducing component count, complexity, and cost; (ii) the second battery 246b in cap functions as a portable power bank, increasing device autonomy; and (iii) the physiotherapy device is ensured to always maintain sufficient charge for therapy, while still supporting convenient external charging through the cap.

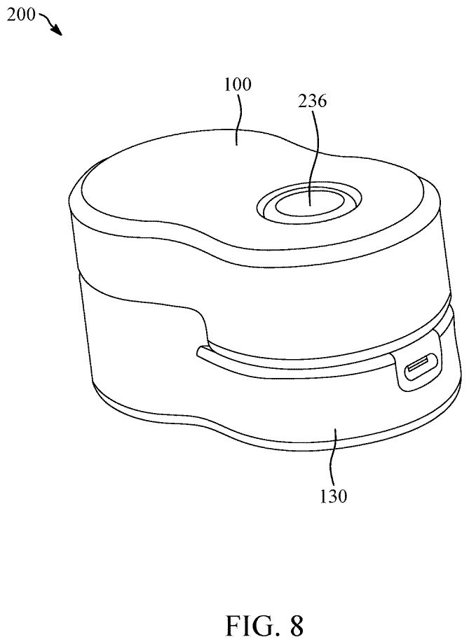

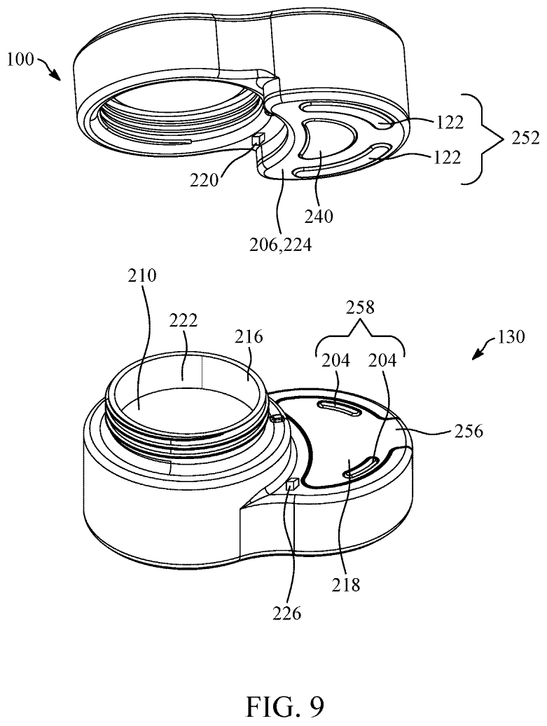

Referring to FIGS. 8 to 12, in an embodiment of the present invention, another configuration of the phototherapy device is provided. Referring to FIGS. 8 and 9, in an embodiment of the present invention, provide a physiotherapy device 200 configured both for storing substances to be applied to the human body and for integrating a therapy module 228 to deliver physical therapy. The dual functionality enables not only the storage and dispensing of application substances but also the promotion of enhanced skin absorption through various therapeutic methods. The therapy may include, but is not limited to, light therapy, microcurrent stimulation, vibration stimulation, hot compress therapy, cold compress therapy, and roller massage. The application substances may include, but are not limited to, eye cream, face cream, essence, facial cleanser, and sunscreen.

The physiotherapy device 200 comprises a liquid container 130 and a cap, which is a housing 100. The liquid container 130 defines a storage space 210 for holding the application substance, and a bottle end 216 provided with a bottle opening 222 in communication with the storage space 210. The housing 100 is attachable to the bottle end 216 and has a closed state, in which it seals the bottle opening 222 to prevent leakage of the stored substance, and an open state, in which the bottle opening 222 is exposed to allow the substance to be dispensed.

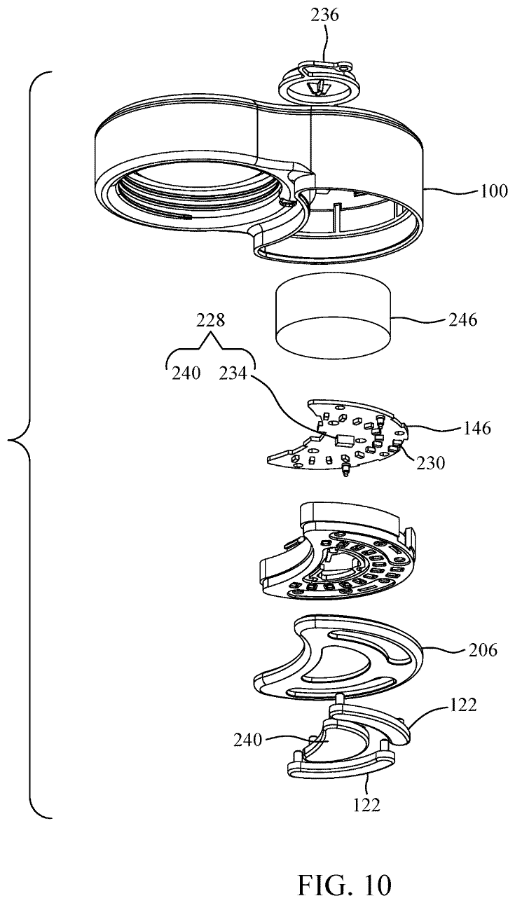

Referring to FIG. 10, the physiotherapy device 200 further comprises the therapy module 228, which is mounted on the housing 100. The therapy module 228 is configured to perform one or more therapeutic functions on the skin, thereby promoting absorption of the applied substance. The therapy module 228 may include one or a combination of: a microcurrent generator, a phototherapy lamp 230, a vibration motor, or a semiconductor heating/cooling element for hot or cold compresses. These components are electronic in nature and require electrical power for operation.

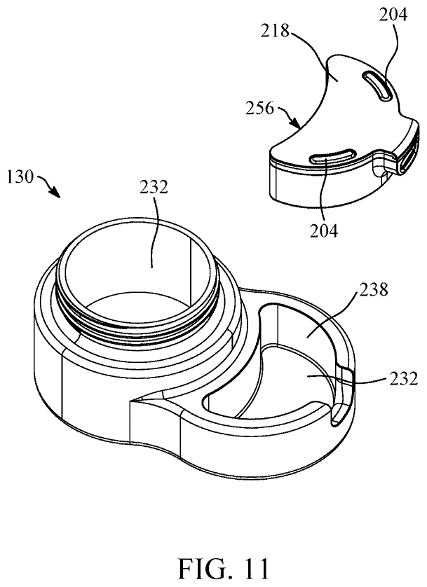

Accordingly, referring to FIGS. 10 and 11, the physiotherapy device 200 further includes a charging device 256, a power storage unit 246, and a second charging module 252. The charging device 256 is fixed to the liquid container 130 and includes a first charging module 258. The power storage unit 246 is disposed within the housing 100 and is configured to supply power to the therapy module 228. The second charging module 252 is electrically connected to the power storage unit 246 and is arranged on the housing 100 such that, when the housing 100 is secured to the liquid container 130, the first charging module 258 and the second charging module 252 are automatically aligned and electrically coupled, enabling charging of the power storage unit 246.

In this configuration, the housing 100 serves not only as a closure for the liquid container 130, but also as an integrated therapeutic device. This dual function eliminates the need for a separate beauty device dedicated to skincare therapy. Similarly, the liquid container 130 functions not only as a container for storing the application substance, but also as a charging base for the power storage unit 246 of the housing 100, eliminating the need for a separate charging base. This design reduces the total number of required components, lowers production costs, improves storage convenience, and prevents accidental loss of a stand-alone charging base.

Furthermore, because the housing 100 and the liquid container 130 are typically used together, the likelihood of either component being misplaced is low. The integrated charging design ensures that the housing 100 can be charged in a timely and consistent manner, without requiring the user to locate and connect a separate charger. When the housing 100 is securely attached to the liquid container 130, the first charging module 258 and second charging module 252 automatically align and engage, ensuring charging occurs whenever the cap is not in use. This maximizes charging opportunities and helps prevent situations in which the therapy module lacks sufficient power. When the housing 100 is opened for use, the electrical connection between the first charging module 258 and the second charging module 252 is automatically disconnected, thereby ensuring safe operation during physiotherapy.

The electrical connection between the first charging module 258 and the second charging module 252 may be implemented through a variety of methods, including but not limited to wired charging and wireless charging.

As shown in FIGS. 10 and 11, the first charging module 258 includes two output electrodes 204, while the second charging module 252 includes two input electrodes 122. When the housing 100 is in the closed state, the two output electrodes 204 are brought into one-to-one correspondence with the two input electrodes 122, thereby establishing an electrical connection for charging the power storage unit 246. The output electrodes 204 and input electrodes 122 may take various structural forms, including but not limited to a Type-C interface, metal plates, metal springs, metal bumps, or spring pins.

The input electrodes 122 serve a dual function of both charging and providing therapeutic stimulation. In a preferred embodiment, the input electrodes 122 also act as therapeutic electrodes for delivering microcurrent stimulation. Specifically, the input electrodes 122, the power storage unit 246, and a circuit board 146 disposed within the housing 100 are electrically connected to form a microcurrent circuit. When the input electrodes 122 come into contact with the skin, an electrical path is formed through the skin, thereby closing the microcurrent circuit among the input electrodes 122, the circuit board 146, the power storage unit 246, and the skin. This allows microcurrent stimulation to be applied directly to the skin.

In an embodiment, the two input electrodes 122 are also configured to participate in a charging circuit. When the input electrodes 122 engage with the output electrodes 204 of the charging device 256, a charging circuit is established among the input electrodes 122, the circuit board 146, the power storage unit 246, and the charging device 256. Accordingly, the system employs a shared set of input electrodes 122 that can alternately function as charging electrodes or as microcurrent electrodes. The microcurrent circuit and charging circuit are designed as independent circuits, such that only one is active at a given time. When the microcurrent circuit is active, the charging circuit is disabled, and vice versa.

This shared-electrode design achieves both charging and therapeutic microcurrent stimulation using a single pair of electrodes. As a result, the number of electrodes required is minimized, the overall structure is simplified, and both production complexity and material costs are reduced.

To facilitate independent switching between the charging circuit and the microcurrent circuit, a control mechanism is provided. In one embodiment, a button 236 or a touch-sensitive switch is electrically connected to the circuit board 146. When the button 236 is actuated, the circuit board 146 receives a trigger signal, enabling the microcurrent circuit while disabling the charging circuit. When the input electrodes 122 are then placed in contact with the skin, the microcurrent loop is completed, thereby delivering microcurrent stimulation to the skin. Conversely, when the circuit board 146 does not receive a trigger signal, it defaults to disabling the microcurrent circuit and enabling the charging circuit. In this state, when the input electrodes 122 engage with the output electrodes 204 of the charging device 256, a charging loop is formed, thereby charging the power storage unit 246.

In an embodiment, the system may include one or more buttons 236 to allow the user to manually and selectively control the activation of the charging circuit and microcurrent circuit. This provides operational flexibility while maintaining circuit independence and ensuring user safety.

In another embodiment, the system may determine whether the input electrodes 122 are in contact with the skin or with the charging device 256 by detecting the resistance value of the electrical connection formed between the two input electrodes 122. Since the electrical resistance of human skin differs significantly from that of the conductive components of the charging device 256, the measured resistance value can be used to reliably distinguish between therapeutic use and charging operation.

In yet another embodiment, a position sensor (not shown) may be provided to determine whether the input electrodes 122 are in contact with the skin or the charging device 256. The position sensor may be configured to detect relative positional information between the liquid container 130 and the housing 100. Based on this detected information, the system can automatically switch between therapeutic functionality and charging functionality.

The input electrodes 122 can also serve as heat conduction elements for applying cold or hot compresses. In this embodiment, the therapy module 228 includes a temperature element 234, which may be a heating element, a cooling element, or a combined thermoelectric element. The housing 100 defines an installation space 248 and two mounting holes 112 communicating with the installation space 248. Each of the two input electrodes 122 is disposed within a respective mounting hole 112. The temperature element 234 is positioned inside the installation space 248 and in direct contact with the input electrodes 122, enabling efficient thermal transfer from the temperature element 234 to the input electrodes 122.

The input electrodes 122 are metallic components exposed on the outer surface of the housing 100, such that, when in contact with the skin, they rapidly transfer heat or cold generated by the temperature element 234 to the skin surface. When the temperature element 234 operates in a cooling mode, the input electrodes 122 deliver a cooling compress to the skin. Conversely, when the temperature element 234 operates in a heating mode, the input electrodes 122 deliver a hot compress. The temperature element 234 may be a semiconductor thermoelectric device, such as a Peltier element, in which the direction of the current determines whether heating or cooling occurs. Alternatively, the temperature element 234 may comprise a heating resistor configured to elevate the temperature of the input electrodes 122.

In an embodiment, the input electrodes 122 may be arranged flush with the outer surface of the housing 100 or may protrude slightly therefrom to improve skin contact. The temperature element 234 may be in direct thermal contact with the inner surface of the input electrodes 122, or coupled via a thermally conductive medium such as silicone grease to enhance heat transfer efficiency. In alternative configurations, the temperature element 234 may be located externally, outside of the housing 100 and installation space 248, while still being thermally coupled to the input electrodes 122.

The input electrode 122 is configured as a floating electrode, movably supported within a mounting hole of the applicator surface. An elastic conductor 134, such as a spring made of conductive metal or a conductive elastomer, may be disposed beneath the electrode. This arrangement enables the electrode to move inward and outward relative to the applicator surface under applied pressure, thereby maintaining consistent contact with the skin while simultaneously preserving electrical continuity. The elastic conductor 134 not only provides mechanical compliance for improved adhesion but also serves as an electrical pathway, ensuring reliable performance during microcurrent therapy and charging operations.

In an embodiment, as illustrated in FIG. 10, the physiotherapy device 200 can further comprise a stimulation element 240 with a stimulation surface 224 configured to provide one or more therapeutic functions. The stimulation element 240 may be configured either as a single integrated functional unit or as a combination of multiple functional components housed within the housing 100 of the physiotherapy device 200. This configuration enables the device to provide one or more therapeutic modalities either simultaneously or in a sequential manner. The combinations of therapeutic effects may include vibration therapy, thermal therapy (heating or cooling), phototherapy, ultrasonic stimulation, piezoelectric stimulation, and microcurrent therapy. The stimulation element 240 may be provided, which transfers heat from the temperature element 234 to the skin, without simultaneously serving as an input electrode 122.

In an embodiment, the housing 100 is provided with two input electrodes 122, one of which doubles as an input electrode 122. The therapy module 228 in this embodiment includes a temperature element 234 that may be disposed inside the other input electrode 122, thereby enabling the input electrode 122 to serve as the stimulation element 240. In this way, the same electrode provides charging, microcurrent stimulation, and various stimulation therapies.

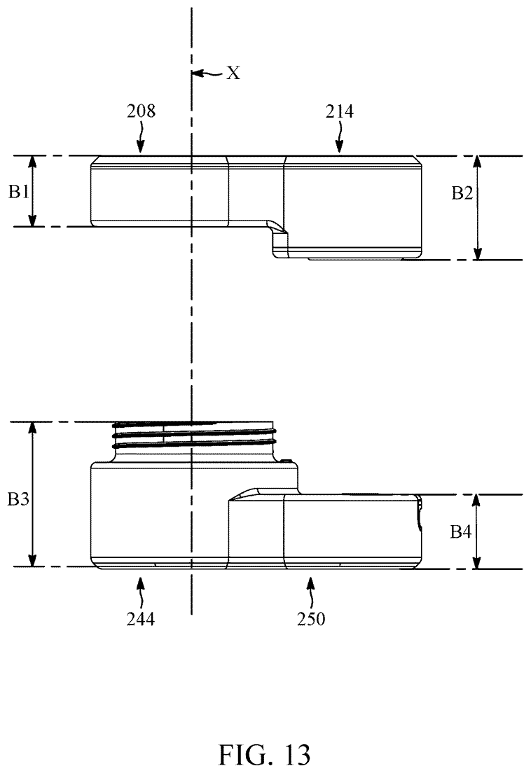

Referring to FIG. 13, in an embodiment, the housing 100 includes a first portion 208 and a second portion 214. The first portion 208 is rotatably connected to the bottle end 216 about a rotation axis X and covers the bottle opening 222. The second portion 214 is disposed eccentrically relative to the rotation axis X. The therapy module 228, the power storage unit 246, and the second charging module 252 are accommodated within the second portion 214. This arrangement allows the entire housing 100 to be rotated by gripping the second portion 214, providing a longer rotation arm that reduces the rotational force required to open or close the cap.

In an embodiment, the first portion 208 is threadedly coupled with the third portion 244 of the liquid container 130. Alternatively, the physiotherapy device 200 may be directly mounted to the third portion 244 through a tight fit. The thickness B1 of the first portion 208 may be less than the thickness B2 of the second portion 214, such that the second portion 214 protrudes toward the liquid container 130 relative to the first portion 208. The second charging module 252 is disposed on the side of the second portion 214 facing the liquid container 130.

The liquid container 130 includes the third portion 244 and a fourth portion 250. The third portion 244 defines the storage space 210, while the fourth portion 250 houses the charging device 256. The height B3 of the third portion 244 is greater than the height B4 of the fourth portion 250. The third portion 244 corresponds to the first portion 208, and the fourth portion 250 corresponds to the second portion 214. In this way, the first portion 208 and second portion 214 of the cap form a stepped surface, while the third portion 244 and fourth portion 250 of the bottle body form a corresponding stepped surface. These stepped surfaces engage in a mutually limiting manner, thereby achieving a positioning effect during installation.

Moreover, the increased thickness of the second portion 214 provides sufficient internal space for accommodating electronic components, such as a phototherapy lamp 230, a circuit board 146, and the power storage unit 246. In some embodiments, both the first portion 208 and the second portion 214 are circular and smoothly transition into one another. As used herein, the term “circular” refers to an approximately circular shape, which may include a quarter-circle, two-thirds circle, or full-circle configuration. Optionally, when viewed from above, the first portion 208 and the second portion 214 together form a figure-eight shape.

Referring to FIG. 9, the housing 100 may further include a first limiting rib 220, and the liquid container 130 may further include a second limiting rib 226. When the cap is closed, the first limiting rib 220 abuts against the second limiting rib 226, thereby restricting further rotation of the housing 100 relative to the liquid container 130. This design not only maintains the sealing performance of the liquid container 130 but also facilitates the positional alignment of the charging structures, specifically, the input electrode 122 and the output electrode 204, during the charging process.

In an embodiment, the second portion 214 is provided with a stimulation element having a stimulation surface 224, and the charging device 256 defines a charging surface 218. The stimulation surface 224 and the charging surface 218 are disposed opposite one another, such that the input electrode 122 is positioned on the stimulation surface 224, while the output electrode 204 is positioned on the charging surface 218.

In an embodiment, the stimulation surface 224 and the charging surface 218 are each oriented perpendicular to the rotation axis of the first portion 208. In other embodiments, these surfaces are disposed parallel to the rotation axis. When the second portion 214 is engaged with the third portion 244 and the two are fully mated, the stimulation surface 224 and the charging surface 218 come into abutment. This not only enables electrical conduction between the output electrode 204 and the input electrode 122, but also prevents further rotational movement of the second portion 214, thereby serving as a rotational limiting structure.

Referring to FIGS. 8 to 10, and FIGS. 14A and 15A, the physiotherapy device 200 comprises a housing 100, a battery 246a, a circuit board 146, and a pair of input electrodes 122. The battery 246a and the circuit board 146 are disposed within the housing 100 and are electrically connected thereto. The pair of input electrodes 122 extends outwardly from the housing 100 so as to contact the skin of a user. The battery 246a, circuit board 146, and the pair of input electrodes 122 are electrically connected to define a microcurrent circuit 160. The battery 246a, circuit board 146, and the pair of input electrodes 122 are further connected to define a charging circuit 170. The microcurrent circuit 160 and the charging circuit 170 are independent from one another.

The microcurrent circuit 160 is configured to establish a microcurrent loop when the input electrodes 122 are in contact with the skin, whereas the charging circuit 170 is configured to establish a charging loop when the input electrodes 122 are in contact with a charging device 256. When the microcurrent loop is active, the charging loop remains inactive, thereby providing only microcurrent stimulation. Conversely, when the charging loop is active, the microcurrent loop remains inactive, thereby providing only charging of the battery 246a.

In this manner, the technical solution of the present invention enables the shared use of the input electrodes 122 for both charging and microcurrent stimulation. As such, it is unnecessary to provide separate electrode pairs for these two functions, thereby reducing the total number of electrodes, simplifying the device structure, and lowering material and production costs. Moreover, since the housing 100 accommodates a reduced number of electrode interfaces, the overall structural integrity of the housing 100 is enhanced.

When the pair of input electrodes 122 is not connected, neither the microcurrent circuit 160 nor the charging circuit 170 is established. Only upon electrical connection of the input electrodes 122 does either the microcurrent circuit 160 or the charging circuit 170 become active. Various approaches may be adopted to selectively and independently activate the microcurrent circuit 160 and the charging circuit 170. In one embodiment, a manual switch, such as a button or a touch-sensitive switch, is provided on the respective circuit for control. In another embodiment, the electrical resistance between the pair of input electrodes 122 may be utilized to determine whether the electrodes are in contact with the skin or the charging device 256. In yet another embodiment, a sensor 154 is disposed within the device to detect parameters indicative of whether the input electrodes 122 are in contact with the skin or the charging device 256, and to control circuit activation accordingly.

Referring to FIGS. 10, 14A, and 15A, in an embodiment, the housing 100 is provided with a trigger button 236, which is electrically connected to the circuit board 146. The circuit board 146 is configured to control the switching of the microcurrent circuit 160 and the charging circuit 170 in response to a trigger signal generated by the trigger button 236. In operation, when the microcurrent circuit 160 is in a connected state, the charging circuit 170 is maintained in a disconnected state. Conversely, when the microcurrent circuit 160 is in a disconnected state, the charging circuit 170 is maintained in a connected state.

In one implementation, the trigger button 236 is realized as a mechanical button or a touch-sensitive switch. When the trigger button 236 is actuated, it generates a trigger signal that is transmitted to the circuit board 146. Upon receipt of the trigger signal, the circuit board 146 controls the microcurrent circuit 160 to enter the connected state while simultaneously controlling the charging circuit 170 to enter the disconnected state. When the pair of input electrodes 122 contacts the skin of the user, an electrical connection is formed through the skin, thereby completing a circuit that includes the battery 246a, the circuit board 146, the input electrodes 122, and the skin, thus delivering microcurrent stimulation.

Moreover, when the circuit board 146 does not receive a trigger signal, it controls the microcurrent circuit 160 to remain disconnected and the charging circuit 170 to be in a connected state. In this condition, when the pair of input electrodes 122 come into contact with the output electrodes 204 of the charging device 256, an electrical connection is formed through the charging device 256, thereby completing a circuit that includes the battery 246a, the circuit board 146, the input electrodes 122, and the charging device 256, thus enabling charging of the battery 246a.

In a further refinement, the microcurrent circuit 160 may be provided with a first switch 162, and the charging circuit 170 may be provided with a second switch 172. The circuit board 146 is configured to control these switches based on the trigger signal. When the trigger signal is received, the circuit board 146 controls the first switch 162 to be conductive and the second switch 172 to be non-conductive, thereby maintaining the microcurrent circuit 160 in a connected state and the charging circuit 170 in a disconnected state. Conversely, when no trigger signal is received, the circuit board 146 controls the first switch 162 to be non-conductive and the second switch 172 to be conductive, thereby maintaining the microcurrent circuit 160 in a disconnected state and the charging circuit 170 in a connected state.

Referring to FIGS. 9 to 11, the physiotherapy device 200 further comprising a stimulation element 240 with a stimulation surface 224 configured to provide one or more therapeutic functions. The stimulation element 240 is electrically connected to the circuit board 146, and its operation is controlled by a trigger button 236. The trigger button 236 functions as a power control element for activating and deactivating the therapeutic functions of the device. When the trigger button 236 is actuated, therapeutic functions such as microcurrent stimulation, light therapy, thermal therapy, magnetotherapy, ultrasonic wave therapy, and vibration stimulation are selectively activated, thereby delivering therapy to the user. When the trigger button 236 is not actuated, the therapeutic functions are deactivated, and charging of the device may proceed uninterrupted.

In an embodiment, the stimulation element 240 comprises one or more of a phototherapy lamp 230, a vibration motor, a piezoelectric element, an ultrasonic wave therapy element, a magnetic element, and a temperature element 234. In the case where the stimulation element 240 includes the phototherapy lamp 230, a portion of the housing 100 is configured as a light-transmitting region 206. The phototherapy lamp 230 is disposed within the housing 100, mechanically secured to the circuit board 146, and oriented toward the light-transmitting region 206. During operation, the phototherapy lamp 230 emits therapeutic light through the light-transmitting region 206, thereby providing phototherapy. Furthermore, the input electrodes 122 and the light-transmitting region 206 are disposed on the same side of the housing 100 such that microcurrent stimulation and phototherapy can be applied simultaneously. In other embodiments, the phototherapy lamp 230 may be mounted directly outside of the housing 100.

In an embodiment, the stimulation element 240 can be a temperature element 234. The temperature element 234 includes a heat conductor. The heat conductor can be a metallic component exposed on the exterior of the housing, and is configured to transfer heat or cold to the skin of the user. The temperature element 234 generates heating or cooling and is thermally coupled to the heat conductor. When the temperature element 234 operates in a cooling mode, the heat conductor delivers a cooling effect to the skin; when the temperature element 234 operates in a heating mode, the heat conductor delivers a heating effect to the skin. In an embodiment, the temperature element 234 is a semiconductor element, wherein reversal of current flow through the semiconductor alters its operation between heating and cooling modes. In another implementation, the temperature element 234 is a heating resistor configured to transfer heat to the heat conductor.

In an embodiment, the temperature element 234 is disposed within the housing 100 and electrically connected to the circuit board 146. The housing 100 includes a mounting hole 112, within which the heat conductor can be embedded. The heat conductor may be positioned flush with the outer surface of the housing 100 or may protrude therefrom. The temperature element 234 can be in direct contact with an inner surface of the heat conductor, or can be coupled thereto via thermal grease to improve heat transfer.

In an embodiment, the housing 100 has two mounting holes 112, and the two input electrodes 122 are mounted therein. To enable simultaneous application of microcurrent stimulation, vibration stimulation, piezoelectric stimulation, magnetic stimulation, ultrasonic wave stimulation, and thermal stimulation, the heat conductor and the input electrodes 122 are disposed on the same side of the housing 100. When the physiotherapy device 200 is applied to the skin, the user's skin simultaneously contacts both the input electrodes 122 and one or more stimulation elements 240. In this manner, microcurrent stimulation may be applied through the input electrodes 122 while a vibration, thermal, piezoelectric, magnetic, or ultrasonic wave therapy effect can be provided via the stimulation element 240. Additionally, the protrusion height of the input electrodes 122 relative to the outer surface of the housing 100 can be substantially equal to the protrusion height of the heat conductor. As such, no substantial height difference exists between the two components, thereby ensuring consistent and simultaneous skin contact for combined therapy. In an embodiment, the stimulation element 240 may alternatively be disposed externally to the housing 100.

In an embodiment, the switching of the microcurrent circuit 160 and the charging circuit 170 is controlled by the circuit board 146 based on the actual resistance measured between the pair of input electrodes 122. When the measured resistance exceeds a first threshold value, the circuit board 146 controls the microcurrent circuit 160 to be in a connected state and the charging circuit 170 to be in a disconnected state. Conversely, when the measured resistance falls below a second threshold value, the circuit board 146 controls the microcurrent circuit 160 to be in a disconnected state and the charging circuit 170 to be in a connected state. Since the electrical resistance of human skin differs substantially from the resistance characteristics of the charging device 256, this resistance differential may be detected and utilized by the circuit board 146 to determine whether the input electrodes 122 are in contact with the skin or with the charging device 256, thereby enabling reliable circuit switching.

In an embodiment, the first threshold is selected according to the resistance characteristics of human skin and may range between approximately 2 kilo-ohms and 20 megaohms. The second threshold may be determined according to the charging voltage and current of the physiotherapy device 200. For example, in a portable physiotherapy device 200, the charging voltage may be between 4.5 V and 18 V, and the second threshold may be set in accordance with the specific charging current corresponding to this voltage range.

In an embodiment, the circuit board 146 is configured to switch the microcurrent circuit 160 and the charging circuit 170 based on an input signal received from the pair of input electrodes 122. Specifically, when the input electrodes 122 are placed in contact with the charging device 256, the charging device 256 delivers a charging signal to the input electrodes 122. Upon detection of this charging signal, the circuit board 146 controls the first switch 162 to be non-conductive and the second switch 172 to be conductive, thereby maintaining the microcurrent circuit 160 in a disconnected state and the charging circuit 170 in a connected state. Conversely, when the input electrodes 122 are electrically connected but no charging signal is received from the charging device 256, the circuit board 146 controls the first switch 162 to be conductive and the second switch 172 to be non-conductive, thereby maintaining the microcurrent circuit 160 in a connected state and the charging circuit 170 in a disconnected state.

Referring to FIG. 11, the liquid container 130 is further provided with a storage cavity 232 for accommodating a charging device 256. In the illustrated embodiment, the fourth portion 250 defines the storage cavity 232, such that the charging device 256 is securely housed within the fourth portion 250 for protection. The fourth portion 250 further includes a through-hole configured to allow a charging port of the charging device 256 to extend outward, thereby enabling connection of a charging cable between the charging port and an external power supply.

The storage cavity 232 is formed with an opening 238 oriented in the same direction as the bottle opening 222. The output electrode 204 of the charging device 256 is exposed through the opening 238, thereby establishing electrical contact with the input electrode 122. In this manner, the charging device 256 may be inserted into, and removed from, the storage cavity 232 through the opening 238. To prevent inadvertent displacement of the charging device 256 from the storage cavity 232, the inner wall of the cavity may be provided with protrusions configured to abut against the charging device 256, restricting movement toward the opening 238. In alternative embodiments, the charging device 256 may be permanently secured to the cavity wall by adhesive bonding, thereby enhancing fixation.

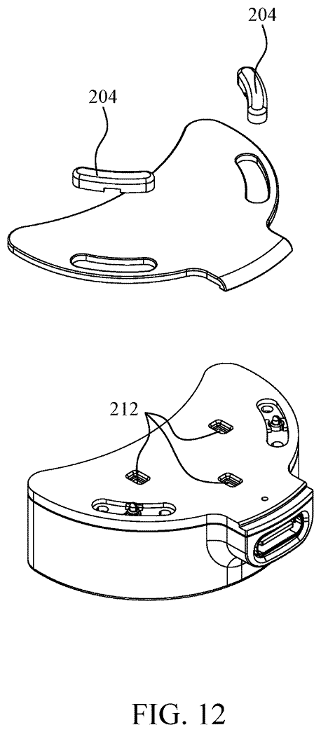

Referring to FIG. 12, in an embodiment, the charging device 256 may be further equipped with an ultraviolet (UV) light source 212. The ultraviolet light source 212 is disposed to face the input electrode 122, and emits germicidal radiation, such as ultraviolet (UV) light, to disinfect the input electrode 122. This ensures hygienic conditions at the input electrode 122 when brought into contact with the user's skin, thereby reducing the risk of skin infection. To enable such functionality, the housing of the charging device 256 is provided with a light-transmitting region 206, through which radiation from the ultraviolet light source 212 passes.

The docking interface between the housing 100 and the charging device 256 may be arranged on a surface disposed either perpendicular or parallel to the rotation axis of the assembly.

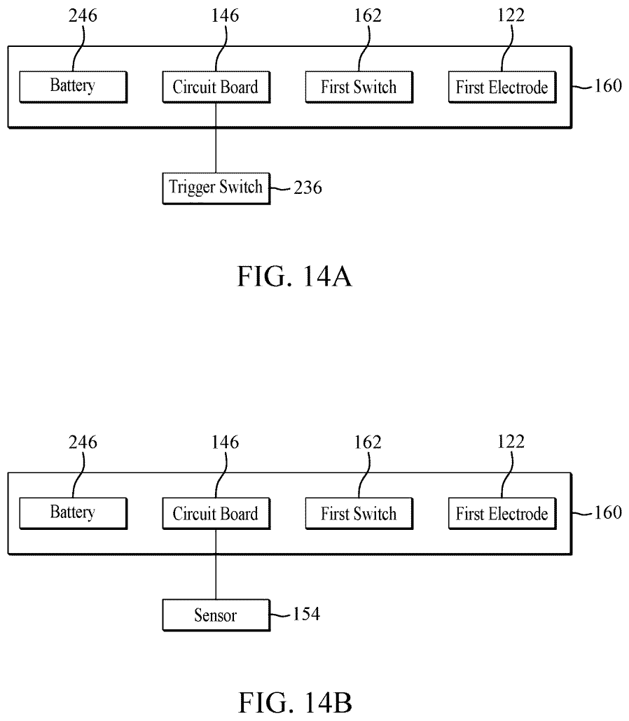

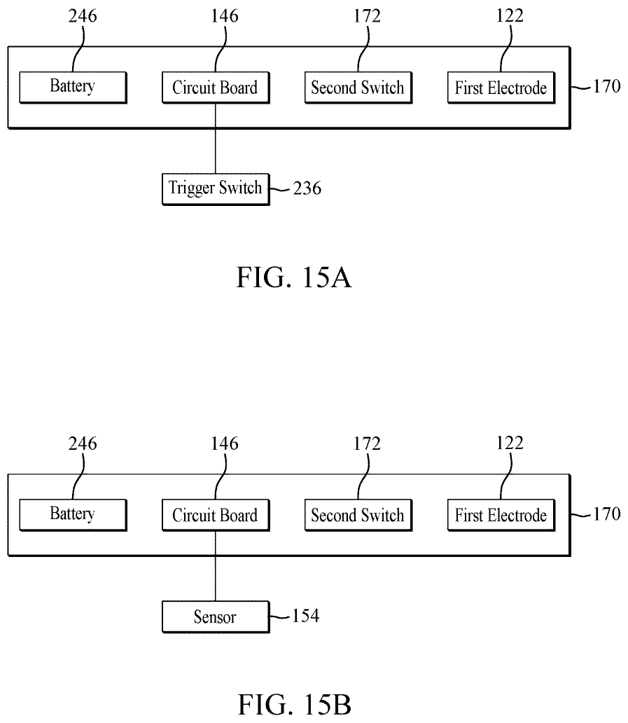

Referring to FIGS. 14B and 15B, the physiotherapy device 200 further comprises a sensor. The circuit board 146 is configured to control the microcurrent circuit 160 and the charging circuit 170 based on data detected by the sensor 125. In an embodiment, the sensor 154 can be a temperature sensor. When the temperature detected by the sensor 154 is greater than or equal to a first preset temperature, the circuit board 146 controls the microcurrent circuit 160 to be in a connected state. When the detected temperature is less than the first preset temperature, the circuit board 146 controls the charging circuit 170 to be in a connected state. Since the normal body surface temperature generally ranges from 36° C. to 37° C., and considering that external factors such as exposure to cold air may reduce the surface temperature, the first preset temperature may be set to approximately 34° C. or 35° C. Thus, when the sensor 125 contacts the skin and detects a temperature greater than or equal to the first preset temperature, this indicates that the input electrodes 122 are in contact with the skin. Accordingly, the circuit board 146 controls the microcurrent circuit 160 to be connected and the charging circuit 170 to be disconnected.

In an embodiment, the sensor 154 can be a position sensor. The circuit board 146 selectively connects or disconnects the microcurrent circuit 160 and the charging circuit 170 based on data received from the sensor 154. For example, when the sensor 125 is realized as a Hall effect sensor, the charging device 256 is provided with a magnet. When the physiotherapy device 200 is docked with the charging device 256, the Hall effect sensor detects a high magnetic field strength. Based on this detection, the circuit board 146 controls the charging circuit 170 to be conductive, thereby enabling charging of the battery 246a. Conversely, when the therapy device 10 is separated from the charging device 256, the Hall effect sensor detects a low magnetic field strength, or no magnetic field at all. Based on this condition, the circuit board 146 controls the microcurrent circuit 160 to be conductive, thereby enabling microcurrent stimulation.

Furthermore, in an embodiment, the physiotherapy device 200 may include a ball bearing rollably mounted to the housing 100, which is configured to provide a rolling massage to the skin. In an embodiment, the input electrodes 122 themselves may be configured as a ball bearing, thereby enabling simultaneous provision of microcurrent stimulation and rolling massage.

In an embodiment, the input electrodes 122 may be directly fixed to the housing 100 so as to prevent movement relative thereto. In such configurations, the input electrodes 122 may take the form of a plate, a cap, or other suitable geometries. For example, when the input electrodes 122 are plate-shaped, they may be directly secured to the outer surface of the housing 100, thereby eliminating the need for the mounting hole 112. Alternatively, when the input electrodes 122 are cap-shaped, the peripheral edge of the electrode cap may be inserted into the mounting hole 112 for secure mounting.

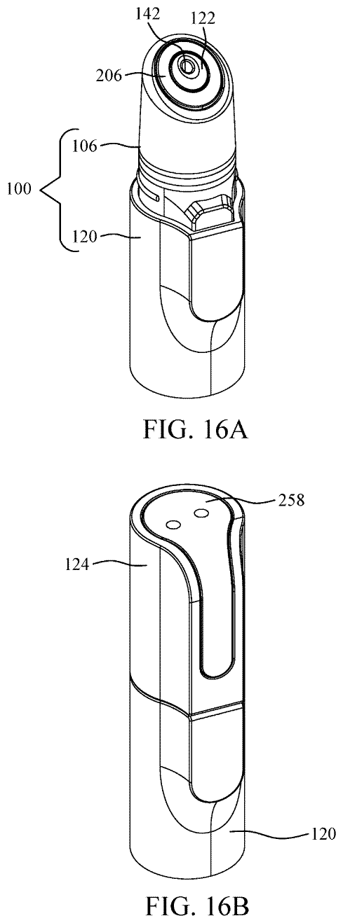

Referring now to FIGS. 16A and 16B show an embodiment of a physiotherapy device 200 comprising a housing 100 and an applicator surface 136 disposed at one end of the housing 100. The housing 100 defines an application surface 136 configured for contact with the user's skin. A pair of input electrodes 122 is mounted on the application surface 136. Each input electrode 122 is supported within a corresponding upper portion 106 and resiliently biased by an elastic conductor housed in the base 120, thereby forming a floating electrode structure that permits axial displacement of the electrodes relative to the applicator head. This floating configuration allows the electrodes to adapt to surface irregularities of the skin, thereby enhancing adhesion and improving uniformity of current conduction.

The input electrodes 122 are operable in a dual-functional manner. When the applicator head is applied to the skin, the input electrodes 122 act as microcurrent therapy electrodes, completing a therapeutic circuit via an internal circuit board (not shown) disposed within the housing 100. The control interface, provided on a side surface of the housing 100, enables a user to selectively activate or adjust the microcurrent therapy. In another mode of operation, the same input electrodes 122 are configured to engage with a pair of corresponding output electrodes provided on a detachable cap 124. When the cap 124 is attached, the electrodes serve as charging output electrodes 204 to receive electrical power for charging an internal power supply of the device.

In some embodiments, the detachable cap includes a power storage unit 246, such as a rechargeable battery, thereby functioning as a portable power bank. This enables the physiotherapy device 200 to be charged directly from the cap, even when the cap 124 is not connected to an external charger. Thus, the dual-purpose electrodes, in cooperation with the cap body, provide both therapeutic and charging functionality without requiring a separate charging port on the device itself, improving convenience and reducing structural complexity.

The physiotherapy device 200 may further include optional therapeutic modules integrated within or proximate to the applicator head. For example, a phototherapy lamp 230 may be disposed within the applicator head for providing phototherapy through the application surface 136. The light-emitting module may comprise one or more LEDs configured to emit light in red, blue, or infrared wavelengths for cosmetic or physiotherapy benefits. In certain embodiments, the input electrodes 122 may be arranged concentrically around the phototherapy lamp 230 to permit simultaneous delivery of microcurrent therapy and light therapy.

The present invention provides significant industrial applicability in the fields of physiotherapy, personal healthcare, and cosmetic treatment. By integrating floating input electrodes capable of dual operation for both charging and microcurrent therapy, the device achieves a compact, multifunctional design that reduces the number of components while enhancing usability. The inclusion of a detachable cap with its own power storage unit functioning as a power bank further increases convenience, enabling the device to be charged in diverse settings without reliance on conventional charging accessories. Additionally, the floating electrode configuration improves skin conformity, ensuring consistent adhesion across different body regions and enhancing therapeutic effectiveness. The device can be manufactured in a wide variety of shapes and sizes to suit professional clinical use, home care, and portable personal wellness applications. Accordingly, the invention addresses unmet needs in the current physiotherapy device industry, offering improved efficiency, safety, and user convenience, while being readily adaptable to large-scale industrial production and commercialization.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended. claims.

1. A physiotherapy device, comprising:
a housing defining an application surface configured to contact skin;
a pair of electrodes mounted on the application surface, each electrode being movably supported by an elastic member to allow axial displacement relative to the housing;
a circuit configured to deliver therapeutic current to the electrodes; and
a control mechanism operable to selectively enable a microcurrent therapy circuit, wherein the electrodes are configured to conform to contour of a skin surface during use.

2. The physiotherapy device of claim 1, wherein each elastic member comprises a spring, a metal strip, or a flexible conductive arm configured to provide resilient displacement of the electrode relative to the housing.

3. The physiotherapy device of claim 1, wherein the pair of input electrodes is floating electrodes movably supported within a mounting cavity and resiliently biased by the elastic member to maintain continuous contact with an uneven skin surface.

4. The physiotherapy device of claim 1, wherein the electrodes each comprise a top wall and a side wall extending along a periphery of the top wall, the side wall defining a positional stop for maintaining the electrode in the housing.

5. The physiotherapy device of claim 1, further comprising a coating surface and a liquid outlet formed on the housing, the liquid outlet being positioned between the pair of electrodes such that liquid applied from the liquid outlet is concurrently delivered with microcurrent stimulation.

6. The physiotherapy device of claim 1, wherein the control mechanism comprises a manual button, touch-sensitive switch, or sensor configured to selectively enable the microcurrent therapy circuit.

7. The physiotherapy device of claim 1, wherein the pair of input electrodes is configured to function as charging electrodes when engaged with a charging base or a cap.

8. A physiotherapy device, comprising:
a housing;
a battery disposed within the housing;
a circuit board electrically connected to the battery;
a pair of input electrodes exposed outside the housing;
wherein the battery, the circuit board, and the pair of input electrodes are electrically connected to form a microcurrent circuit and a charging circuit, the microcurrent circuit and the charging circuit being independent from one another; and
wherein the microcurrent circuit is configured to deliver microcurrent stimulation and the charging circuit is configured to charge the battery.

9. The physiotherapy device of claim 8, further comprising a trigger switch connected to the circuit board, the trigger switch is configured to generate a trigger signal to the circuit board to allow the microcurrent circuit to enter in a connected state while simultaneously controlling the charging circuit to enter in a disconnected state.

10. The physiotherapy device of claim 8 further comprising a first switch connected to the microcurrent circuit and a second switch connected to the charging circuit; and
wherein the circuit board is configured to control the first switch in a connected state and the second switch in a disconnected state upon receiving a trigger signal.

11. The physiotherapy device of claim 8 further comprising a sensor to detect at least one parameter of a user's skin; and wherein the circuit board is configured to control the microcurrent circuit in a connected state and the charging circuit in a disconnected state when the at least one parameter exceeds above a threshold value.

12. The physiotherapy device of claim 8, further comprising one or more stimulation element selected from a group consisting of but is not limited to a phototherapy lamp, a vibration motor, a hot compress module, and a cold compress module.

13. The physiotherapy device of claim 8, wherein the housing defines an applicator surface and a mounting cavity for accommodating the pair of input electrodes, and the pair of input electrodes is movably supported by an elastic conductor, forming floating electrodes configured to maintain contact with a user's skin.

14. The physiotherapy device of claim 8, further comprising a detachable cap including the pair of output electrodes, wherein the pair of input electrodes engage with the pair of output electrodes when the detachable cap is mounted to enable charging of the battery.

15. The physiotherapy device of claim 8, further comprising a liquid container defining a storage cavity for a skin care product, wherein the housing is attachable to the liquid container, and the pair of input electrodes is disposed on an applicator surface for simultaneous delivery of the skin care product and microcurrent therapy.

16. A method for delivering physiotherapy or skin care treatment to a user, comprising:
providing a physiotherapy device comprising a housing defining an application surface, a pair of input electrodes mounted on the application surface, and a circuit electrically connected to the pair of input electrodes;
placing the application surface in contact with a user's skin;
enabling a microcurrent therapy circuit via a control mechanism;
causing the pair of input electrodes to conform to the contour of a user's skin; and
delivering therapeutic current through the pair of input electrodes to the user's skin.

17. The method of claim 16, wherein the pair of input electrodes is movable, supported by an elastic member comprising a spring, a metal strip, or a flexible conductive arm, such that the electrodes are axially displaceable relative to the housing.

18. The method of claim 16, further comprising activating the control mechanism via a manual button, touch-sensitive switch, or sensor to selectively enable the microcurrent therapy circuit.

19. The method of claim 16, further comprising establishing a charging circuit when the electrode engages with a pair of output electrodes of a detachable cap or a charging base, independently of microcurrent therapy.

20. The method of claim 16, further comprising moving the electrode axially via an elastic conductor such that the electrode maintains continuous contact with an uneven skin surface during treatment.
